(19) United States
(12) Patent Application Publication
(10) Pub. No.: US 2021/0140936 A1
(43) Pub. Date: May 13, 2021

(54) POLLUTION MEASURING SENSOR HOUSING

(71) Applicant: SERDAR PLASTÍK SANAYÍ VE TÍCARET ANONÍM SÍRKETÍ, Sincan/Ankara (TR)

(72) Inventor: Serdar Mustafa TÜTEK, Sincan/Ankara (TR)

(73) Assignee: SERDAR PLASTÍK SANAYÍ VE TÍCARET ANONÍM SÍRKETÍ, Sincan/Ankara (TR)

(21) Appl. No.: 16/618,154

(22) PCT Filed: May 17, 2018
(86) PCT No.: PCT/TR2018/000046

§ 371 Date: Nov. 28, 2019

(30) Foreign Application Priority Data
May 30, 2017 (TR) 2017/07951

(51) Int. Cl. G01N 33/18; A47L 15/42; B29C 45/16

(52) U.S. Cl. CPC G01N 33/18; B29L 2031/265; B29C 45/1671; A47L 15/4297

(57) ABSTRACT

Present invention relates to sensor housing which is used in all dishwashers, and providing full sealing within its structure from 4 points by means of TPV 2K injected (2) sealing gasket that is injected via 2K injection wherein sensor providing a possibility to measure the water pollution doesn't require an additional component for a full sealing.

Present invention relates to sensor housing which is used in all white appliances such as dishwashers etc. and providing sealing within its structure wherein sensor providing a possibility to measure the water pollution doesn't require an additional component for a full sealing.

### TECHNICAL FIELD OF THE INVENTION

The present invention relates to the housing in which the sensor measuring pollution of water used by the machine is installed and to sealing property of this housing in dishwashers and all similar white appliances.

### STATE OF THE ART

There is a sensor measuring the pollution of water in dishwashers and all similar white appliances. The sealing of the housing in which this sensor is installed is provided by an additional O-ring and in this case, there may be leakage because of deformation of the O-ring during mounting or over time. When there is a leakage, firstly the sensor doesn't function properly and goes fully out of order after some time.

### BRIEF DESCRIPTION OF THE INVENTION

The subject matter of the present invention is pollution measuring sensor housing; wherein O-rings attached afterwards for sealing in present housings are deformed and lose their sealing capability, wherein the subject matter relates to providing complete sealing from 4 points by means of injecting TPV on the housing.

The present invention relates to the housing in which the sensor used in dishwashers and all similar white appliances is installed, and wherein it relates to providing complete sealing by injecting a second thermoplastic via 2K injection on the body without requiring any additional component.

It is characterized by injecting on the body and by producing via 2K injection on the body unlike the existing gasket systems.

### DETAILED DESCRIPTION OF THE INVENTION

The subject of the invention relates to pollution sensor housing used in dishwashers and all similar white appliances and determining dirty water.

The subject matter of the present invention is pollution measuring sensor housing; wherein O-rings attached afterwards for sealing in present housings are deformed and lose their sealing capability, wherein the subject matter relates to providing complete sealing from 4 points by means of injecting TPV on the housing.

The present invention relates to the housing in which the sensor used in dishwashers and all similar white appliances is installed, and wherein it relates to providing complete sealing by injecting a second thermoplastic via 2K injection on the body without requiring any additional component such as O-ring etc. sealing members.

### BRIEF DESCRIPTION OF THE FIGURES

FIG. 1—is Perspective View of the Pollution Measuring Sensor Housing

FIG. 2—is detailed view of the sealing gasket of the Pollution Measuring Sensor Housing

### LIST OF REFERENCES

1—Turbidity body
2—2K sealing gasket
3—Sealing gasket connecting tabs

In FIG. 1, assembly view of the pollution measuring sensor is shown. Pollution measuring sensor is installed in the water tank in dishwashers etc. white appliances. The functionality of the sensor housing is to protect the sensor measuring the pollution of water in the tank from the water inside the water tank. The mounting is conducted by the connecting tabs (3) of the water tank engaging the tab housings on the sensor housing (1) shown in the picture.

In FIG. 2, detail of the sealing gasket (2), which is produced by 2K injection, of the pollution measuring sensor housing is shown. In existing systems, an additional sealing member such as gasket, O-ring etc., whereas the present invention provides complete sealing from 4 points with the gasket located within the body. It provides 4 points sealing together with gasket lower surface and side surface that are fitted to the area where it is mounted to the water tank in addition to two sealing tie pieces shown in the Figure.

It has the body (1) used in dishwashers etc. all white appliance and determining the dirty water and has TPV (soft thermoplastic elastomer) 2K injection (2) injected on this body (1) unlike the existing sealing gaskets. When the body (1) is produced by injection, softer TPV (soft thermoplastic elastomer) is injected on it before removing from the mold thereby obtaining a single piece product made of two different material.

What is claimed is:

1—The sensor housing used in dishwashers etc. all white appliances and determining the polluted water; characterized in that it comprises gasket (2) providing complete sealing from 4 points within the body (1).

2—The sensor housing according to claim 1, characterized in that it comprises TPV 2 injected sealing gasket (2) providing a complete sealing rather than an additional sealing gasket and injected on the body (1) via 2K injection.
